(19) United States
(12) Patent Application Publication
(10) Pub. No.: US 2018/0133329 A1
(43) Pub. Date: May 17, 2018

(54) COMPOSITIONS AND METHOD FOR STABILIZING A PHARMACEUTICAL FORMULATION, AND METHODS OF MAKING AND USING THE SAME

(71) Applicants: Xin WEN, Alhambra, CA (US); Sen WANG, Alhambra, CA (US)

(72) Inventors: Xin WEN, Alhambra, CA (US); Sen WANG, Alhambra, CA (US)

(73) Assignee: The Board of Trustees of the California State University, Long Beach, CA (US)

(21) Appl. No.: 15/807,405

(22) Filed: Nov. 8, 2017

Related U.S. Application Data
(60) Provisional application No. 62/419,246, filed on Nov. 8, 2016.

(51) Int. Cl. A61K 47/42; A61K 47/26; A61K 9/19

(52) U.S. Cl. CPC A61K 47/42; A61K 9/19; A61K 47/26

(57) ABSTRACT

The disclosure pertains to compositions to stabilize nutrient, food, pharmaceutical, and biological formulations by preventing the crystallization or precipitation of carbohydrates used in the formulations. The compositions contain (i) one or more carbohydrates, sugars, sugar alcohols, analogs thereof, and/or derivatives thereof and (ii) one or more antifreeze proteins (AFPs), mimetics thereof, and/or analogs thereof and methods of making and using the same. The AFP may be selected from antifreeze polypeptides and antifreeze peptides, analogs and mimetics of antifreeze proteins, active fragments of such antifreeze proteins, polypeptide and peptide mimetics, antifreeze peptoids and polymers, and combinations thereof. The carbohydrate, sugar or sugar alcohol has the formula CₘHₙOₚ, where m is an integer of 5 or 6, n is an integer of 2m or 2m+2, and p is equal to m, or the formula CₐH_{b}O_{c}, where a is an integer of 10-12 or 15-18, b is 2a−2 when a is an integer of 10-12 or 2a−4 when a is an integer of 15-18, and c is a−1 when a is an integer of 10-12 or a−2 when a is an integer of 15-18. The AFP, AFP analog, or AFP mimetic inhibits or controls crystallization of the carbohydrate (e.g., during the storage of the solution of the carbohydrate or the analog or derivative thereof, during the freeze-drying of a formulation using the carbohydrate or the analog thereof) and stabilize the solution or the formulation using the carbohydrate or the carbohydrate analog or derivative.

### RELATED APPLICATION

This application claims the benefit of U.S. Provisional Application No. 62/419,246, filed Nov. 8, 2016, incorporated herein by reference in its entirety.

### FIELD OF THE INVENTION

The present invention generally relates to the field of pharmaceutical formulations and methods of stabilizing the same. More specifically, embodiments of the present invention pertain to compositions containing (i) one or more carbohydrates, sugars and/or sugar alcohols and (ii) one or more antifreeze proteins, and methods of making and using the same (e.g., to stabilize a pharmaceutical formulation).

### DISCUSSION OF THE BACKGROUND

Mannitol is a naturally occurring sugar alcohol. It can be prepared commercially by the reduction of dextrose. Although virtually inert metabolically in humans, it occurs naturally in fruits and vegetables. The structure of mannitol is shown in FIG. 1.

Mannitol, mannitol analogs (e.g., substances containing the structure of mannitol or a structure similar to mannitol), and mannitol derivatives are widely used in the food and pharmaceutical industries, as well as in medical practice for a variety of indications. For example, mannitol is a sweetener, a widely-used excipient in various pharmaceutical products, and an FDA-approved drug. For example, mannitol is effective in the promotion of diuresis, in the prevention and/or treatment of renal failure, in reducing elevated intraocular pressure, and in reducing intracranial pressure and treating cerebral edema. Mannitol is an obligatory osmotic diuretic. Mannitol also has veterinary uses.

Mannitol, mannitol analogs, and mannitol derivatives have also been used to protect functional foods and nutrients. For example, mannitol protects probiotic bacteria during storage (see, e.g., Dianawati et al., Role of Calcium Alginate and Mannitol in Protecting Bifidobacterium (2012), Vol. 78, No. 19, 6914-6921). However, the degree of crystallization of mannitol directly relates to the destabilization of the products. The higher the crystallinity of mannitol in the products, the less active are the bioactive ingredients.

FIG. 2 shows a label from a commercially available mannitol solution used for medical and/or pharmaceutical purposes. 20% Mannitol injection USP is a sterile, nonpyrogenic solution in a single dose container for intravenous administration.

There are problems when using mannitol and its analogs and derivatives. For example, solutions of mannitol and its analogs and derivatives may crystallize when exposed to low temperatures. Concentrations greater than 15% have a greater tendency to crystallize. Solutions of mannitol for pharmaceutical and veterinary uses must be inspected for crystals prior to administration or use. If crystals are observed, the container of the mannitol solution should be warmed by appropriate means to a temperature of 60° C. or lower, shaken, then cooled to body temperature before administering. If all crystals are not completely re-dissolved, the container may be rejected. Alternatively, the administration may be set with a filter (e.g., the solution may be filtered before or during use).

Preparation of dried formulations by freeze-drying is widely used in the pharmaceutical and food industries. For example, mannitol is a widely-used excipient in freeze-drying (e.g., to make freeze-dried pharmaceutical formulations to stabilize various biomolecules, such as proteins, enzymes, hormones, vitamins, etc.) and in lyophilization. It serves as a bulking agent in freeze-drying. The presence of mannitol may have many advantages in stabilizing the formulation (e.g., maintaining the rigid structure of the volume of the freeze-dried product, adjusting the isotonicity of the solution, etc.). However, when mannitol is the predominant excipient in the composition of a freeze-dried formulation, it is most often in crystalline form. The crystallization of mannitol during the freeze-drying process changes the distribution of water in the matrix of the freeze-dried product, and can also cause vial breaking, which greatly destabilizes the freeze-dried product. Vial breakage is an issue when using mannitol-containing solutions in freeze-drying and lyophilization, where vial strain and breakage is associated with the crystallization of solutes and/or the crystallization of water. FIGS. 3A-B show examples of such broken vials. Controlling the conditions of the freeze-dry process (e.g., control of annealing) to reduce or avoid crystallization and/or vial breaking can be difficult.

Alternatively, additives (e.g., salts or polyols with structures similar to mannitol) can be used (e.g., as 5% weight-to-volume [w/v] solution). However, such solutions can be very inefficient, difficult to control, and/or expensive. For example, certain additives (e.g., salts, alditols, polyvinyl pyrrolidone, alpha-cyclodextrin, polysorbate 80) may be added to the solution containing mannitol to inhibit mannitol crystallization. The amount of known additives for inhibiting mannitol crystallization are typically between 1-10% w/v.

Antifreeze proteins (AFPs) have been found in many cold-adapted organisms, such as fishes, insects, plants, bacteria, and fungi. AFPs can bind to small ice crystals to inhibit or slow growth and/or recrystallization of ice that would otherwise be fatal to such organisms. Some AFPs are glycoproteins, which are also called antifreeze glycoproteins (AFGPs). AF(G)Ps exhibit remarkable structural and sequential variation among species, and AF(G)Ps often occur as a series of isoforms in a species without close similarities to any other known protein. A few examples are given below. Type I AFPs are small amphipathic α-helical proteins containing periodically repeating amino acids and their molecular weights are about 3-5 kDa. AF(G)Ps mainly consist of repeat units of two or three amino acids, one of them glycosylated, and AF(G)Ps usually have 4 to 32 repeat units and have a molecular weight from 2 kDa to 34 kDa. There are at least 13 known AFPs from Dendroides canadensis (DAFPs) containing varying numbers and sizes of repeat units with sizes of 7-17 kDa. Type II and Type III AFPs are globular proteins without repeating units and they consist of varied secondary structures. Their molecular weights are about 14-16 kDa and about 6 kDa, respectively. Although there are a variety of AF(G)P structures, they are all defined by their ability to bind to ice and inhibit or slow ice growth. Therefore, AF(G)Ps are also called ice-binding proteins. Mimetics of AF(G)Ps have been synthesized, and various engineered AF(G)Ps, fragments of AF(G)Ps, active mimetic fragments of AF(G)Ps have been reported. Natural or engineered AF(G)Ps, active fragments of AF(G)Ps, mimetics of AF(G)Ps, their active mimetic fragments, and combinations thereof can bind to ice crystals to inhibit or reduce growth and/or recrystallization of ice. By binding to ice crystal surfaces, AF(G)P can depress the freezing point of water without apparently affecting the melting point of water. The difference between the depressed freezing point and the melting point is termed thermal hysteresis (TH), and the value of TH is usually a measure of the antifreeze activity of AFPs. AFPs from insects are generally more active than AFPs from other species.

Recently, new roles for AF(G)Ps in controlling the crystallization of non-ice-like compounds (ice-like compounds include ice and gas hydrates) have been reported (see, e.g., Xin Wen and Sen Wang, Nucleoside crystals, crystal nucleation and growth control with antifreeze proteins, U.S. Pat. No. 9,394,327 B1; Chemical & Engineering News (2014), Vol. 92, Iss. 26, p. 22; Wang et al., “Expanding the molecular recognition repertoire of antifreeze polypeptides: effects on nucleoside crystal growth,” Chem. Commun. 48:11555-11557 [2012]). Moreover, the correlation between the antifreeze activity of AFPs and their effects on controlling the crystallization of non-ice-like compounds have been demonstrated (see, e.g., Wang, S. et al., Molecular Recognition of Methyl α-D-Mannopyranoside by Antifreeze (Glyco)Proteins, J. Am. Chem. Soc. [2014], 136: 8973-8981).

U.S. Pat. Appl. Publ. No. 2016/0265073 discloses a method of preventing precipitation of a carbohydrate (exemplified by trehalose) in a solution by one of four particular AFPs, where the mass ratio of the antifreeze protein to the carbohydrate is between about 1:8000 and about 1:30. The four AFPs are identified in U.S. Pat. Appl. Publ. No. 2016/0265073 by at least part of their amino acid sequences.

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY OF THE INVENTION

Embodiments of the present invention relate to a composition comprising (i) one or more carbohydrates, sugars and/or sugar alcohols (and analogs and derivatives thereof) and (ii) one or more antifreeze proteins (AFPs), and methods of making and using the same (e.g., to stabilize a functional food formulation or a pharmaceutical formulation, for example during the storage of the formulations or during a freeze-drying process). The composition comprises an antifreeze protein (AFP) and a carbohydrate.

In one aspect, the AFP is selected from the group consisting of fish AFPs (e.g., type I AFPs, type II AFPs, type III AFPs, type IV AFPs), plant AFPs, insect AFPs, bacteria AFPs, fungi AFPs, fish, plant and insect antifreeze glycoproteins (AFGPs), antifreeze polypeptides and peptides, active fragments of AFPs, AFGPs, antifreeze polypeptides and antifreeze peptides, mimetics of AFPs, AFGPs, antifreeze polypeptides and antifreeze peptides, active fragments of antifreeze protein, glycoprotein, polypeptide and peptide mimetics, and combinations and analogs thereof. In other examples, the AFP may have an amino acid sequence of the formula R¹-(AA1-AA1-AA2)ₓ-R². Each AA1 is independently Ala, Asn, Gly, Val, Leu Pro, Phe, Thr, Tyr, or Ile, each AA2 is independently Thr or Ser or Tyr, R¹ is H, C₁₋₆ alkyl, R³—C(═O)— or R³—OC(═O)—, x is an integer of at least 3, R² is OH, C₁₋₆ alkoxy, R⁴—NH— or R⁴₂N—, R³ is C₁₋₆ alkyl, C₆₋₁₀ aryl, or C₁₋₆ alkyl or C₆₋₁₀ aryl substituted with one or more halogen atoms and/or C₁₋₄ alkyl groups, and R⁴ is C₁₋₄ alkyl. The DAFP isoforms include proteins having an amino acid sequence of the formula A-(Thr-X-Thr-Y)_{z}-B, where A is a sequence of at least 1 amino acid (e.g., Gln-Cys-Thr-Gly-Gly-Ser-Asp-Cys-Ser-Ser-Cys), X is a sequence of from 1 to 3 amino acids and which may include Cys, Y is a sequence of 1-12 amino acids, z is an integer of from 3 to 32, and B is a sequence of 1-5 amino acids (e.g., Gly-Cys-Pro). The AFP or AFGP (AF[G]P) may be natural, engineered, or synthesized. The antifreeze protein, glycoprotein, polypeptide and peptide analogs may have (1) an amino acid sequence that differs from the amino acid sequence of the native protein, glycoprotein, polypeptide or peptide by a limited number (e.g., the number is 30% or less than the total number of amino acids in the sequence, such as 5 or 10 amino acids) amino acids, but that contains any amino acid residues (such as arginines in the case of DAFPs) responsible for thermal hysteresis or other antifreeze activity and (2) at least the same or similar (e.g., >50% of the) antifreeze activity as the native protein, glycoprotein, polypeptide or peptide. The antifreeze protein, glycoprotein, polypeptide and peptide analogs may also include those with the same amino acid sequence of the native protein, glycoprotein, polypeptide or peptide, but modified with non-amino acid substitutions and/or end groups (e.g., amide or ester groups on a carboxylic acid, carboxyl groups on an amine, thiol or alcohol, one or more alkyl groups on an amide, etc.). Antifreeze protein, glycoprotein, polypeptide and peptide analogs may also include those disclosed in U.S. Pat. No. 9,394,327, the relevant portions of which are incorporated herein by reference, and antifreeze peptoids and polymers such as those disclosed in International Pat. Publ. No. WO 2017/066454 and in Mitchell, D. E., et al., “Antifreeze Protein Mimetic Metallohelices with Potent Ice Recrystallization Inhibition Activity,” J. Am. Chem. Soc. (2017) 139:29, 9835-9838, the relevant portions of which are incorporated herein by reference. The AFP, AFP analog, or AFP mimetic inhibits or controls crystallization of the carbohydrate (e.g., during the storage of the solution of the carbohydrate or the analog thereof, during the freeze-drying of a formulation using the carbohydrate or the analog thereof) and stabilizes a solution or formulation including the carbohydrate or the carbohydrate analog.

The carbohydrate is selected from the group consisting of sugars, sugar alcohols, disaccharides and trisaccharides. The sugars and sugar alcohols have the formula CₘHₙOₚ, where m is an integer of 5 or 6, n is an integer of 2m or 2m+2, and p is equal to m, and the di- and trisaccharides have the formula CₐH_{b}O_{c}, where a is an integer of 10-12 or 15-18, and b is 2a−2 when a is an integer of 10-12 or 2a−4 when a is an integer of 15-18, and c is a−1 when a is an integer of 10-12 or a−2 when a is an integer of 15-18. In this aspect, the AFP and the carbohydrate are present in a mass ratio of from about 1:500,000 to about 1:100. In some embodiments, the carbohydrate is the sugar or sugar alcohol of the formula CₘHₙOₚ. Specifically, the carbohydrate may be mannitol, a mannitol analog (e.g., a substance contains at least a mannitol structure or a structure similar to mannitol [for example, a compound having the carbon-oxygen framework or skeleton of mannitol, such as an ether, an ester or a metal salt of mannitol, or a carboxylic acid analog, such as alginic acid and salts thereof]), or a mannitol derivative (e.g., a polymer of mannitol and/or a mannitol analog).

In another aspect, the composition comprises an insect AFP (e.g., a DAFP such as DAFP-1 (SEQ ID NO 1), DAFP-2 (SEQ ID NO 2), DAFP-4 (SEQ ID NO 3) or DAFP-6 (SEQ ID NO 4), or a DAFP isoform) and the sugar or sugar alcohol of the formula CₘHₙOₚ, where m is an integer of 5 or 6, n is an integer of 2m or 2m+2, and p is equal to m. The insect AFP and the sugar or sugar alcohol are present in a mass ratio of from about 1:500,000 to about 1:1000. In either this aspect or the aspect in the previous paragraph, the sugar or sugar alcohol may be mannitol. The insect AFP may also include a DAFP isoform having at least 3 Thr-X-Thr units (e.g., 4-6 such units), where X is a sequence of from 1 to 3 amino acids and which may include Cys. The DAFP isoforms include proteins having an amino acid sequence of the formula A-(Thr-X-Thr-Y)_{z}-B, where A is a sequence of at least 1 amino acid (e.g., 8-12 amino acids, such as Gln-Cys-Thr-Gly-Gly-Ser-Asp-Cys-Ser-Ser-Cys), Y is a sequence of 1-12 amino acids (see, e.g., SEQ ID NOS 1-4), z is an integer of from 3 to 32, and B is a sequence of 1-5 amino acids (e.g., Gly-Cys-Pro).

In further embodiments, the mass ratio of the AFP to the carbohydrate or the insect AFP to the sugar or sugar alcohol is from 1:500,000 to 1:5000 (or higher). For example, the mass ratio of the AFP to the carbohydrate may be from 1:5000 to 1:100, and the mass ratio of DAFP to the sugar or sugar alcohol may be from 1:500,000 to 1:10,000.

When the AFP is one or more of the AFPs or AFGPs having the amino acid sequence of the formula R¹-(AA1-AA1-AA2)ₓ-R², each AA1 may independently be Ala, Asn, Gly, Val, Leu, Pro, Phe, Thr, Tyr, or Ile, and the amino acid sequence may have the formula R¹-[Ala-Ala-Thr]ₓ-R², where 0-10% of the alanines in the formula R¹-[Ala-Ala-Thr]ₓ-R² are replaced with glycine or leucine and 0-10% of the threonines in the formula R¹-[Ala-Ala-Thr]ₓ-R² are replaced with serine or tyrosine. Furthermore, x may be 3-100, 10-60, or 30-50 and/or 10-12.

The AFPs or AFGPs having the amino acid sequence of the formula R¹-(AA1-AA1-AA2)ₓ-R² may include one or more mono- or disaccharides (any of which may further include an acetylamido or other C₁₋₄ alkanoylamido groups in place of an OH group) linked to a threonine (or serine or tyrosine) hydroxyl group through a glycosidic linkage.

A further aspect of the present invention relates to a formulation comprising the present composition in a pharmaceutically acceptable liquid excipient. The pharmaceutically acceptable liquid excipient may comprise deionized and/or distilled water (e.g., a biologically-compatible and/or isotonic saline solution). When the carbohydrate or the sugar or sugar alcohol is mannitol, the mannitol may be present in the pharmaceutically acceptable liquid excipient in an amount of from 1% to about 20% weight-to-volume. The formulation may further comprise a pharmacologically active or biologically active compound.

Adding an AFP or AFGP to a formulation (e.g., for a carbohydrate solution, freeze-drying or lyophilization), the crystallization (e.g., of the carbohydrate, sugar, sugar alcohol and/or water) can be controlled and/or vial strain/breakage can be prevented. In addition, mannitol tends to form crystals, and different crystalline modifications, which may also negatively impact storage stability. By adding an AF(G)P to the formulation, the extent of crystallization and the modification of the mannitol (e.g., its crystalline form) can be altered, and the storage stability of the formulation (e.g., a pharmaceutical formulation including a drug) can be prolonged.

An even further aspect of the present invention relates to a method of inhibiting or preventing crystallization of a carbohydrate, comprising combining an AFP or an AFP analog with a solution of the present carbohydrate (or sugar or sugar alcohol) in a mass ratio of the AFP to the carbohydrate of from about 1:500,000 to about 1:100 to form a mixture, and storing the mixture at a temperature at which the carbohydrate can crystallize in the absence of the AFP for a minimum length of time. The minimum length of time may be 1 day, 1 week, 1 month, 1 year or longer. The solution may (further) comprise deionized and/or distilled water (e.g., a biologically-compatible and/or isotonic saline solution), and the carbohydrate or the sugar or sugar alcohol may be mannitol. The mannitol may be present in the deionized and/or distilled water in an amount of from 1% to about 25% weight-to-volume.

A still further aspect of the present invention relates to a method of freeze-drying a pharmaceutical formulation, comprising the present method of inhibiting or preventing crystallization of a carbohydrate or its derivative, combining a pharmacologically active or biologically active substance with at least a solvent of the solution such that, after performing the method of inhibiting or preventing crystallization of a carbohydrate or its derivative and combining the pharmacologically active or biologically active substance, a pharmaceutical solution is formed, and freeze-drying the pharmaceutical solution to form the pharmaceutical formulation.

One of the purposes of the present invention is to inhibit or prevent mannitol, its analogs or derivatives crystallization for periods of time on the order of months or possibly years. However, some of the AFPs disclosed herein (e.g., DAFPs, and fish AFGPs) inhibit or prevent crystallization of a number of other carbohydrates. Furthermore, the AFP-to-carbohydrate ratio in the present formulation is lower than prior compositions for inhibiting carbohydrate crystallization and the resulting crystallization inhibitory effects are more significant. These and other advantages of the present invention will become readily apparent from the detailed description of various embodiments below.

### BRIEF DESCRIPTION OF THE DRAWINGS

FIG. 1 shows the structure of mannitol.

FIG. 2 is a label from a commercially available pharmaceutical formulation of mannitol for injection.

FIG. 3 shows examples of broken vials resulting from a conventional freeze-drying process.

FIG. 4 shows the results of a series of experiments demonstrating the effectiveness of the present composition and method for inhibiting of preventing the crystallization of mannitol from a concentrated or supersaturated solution thereof.

FIGS. 5A-C show the crystallization of mannitol from a solution of mannitol using seed crystals, in the absence (FIG. 5A) or presence (FIGS. 5B-C) of either of two different AFPs, respectively.

FIG. 6 shows the faces to the 3D structure of type I AFPs.

FIGS. 7A-B show representative CP MAS ¹³C solid-state NMR spectra of D-mannitol from different samples.

FIGS. 8A-C show representative FT-IR spectra of D-mannitol from different samples.

FIG. 9 shows overlays of sample X-ray powder diffraction (pXRD) patterns of crystalline mannitol from lyophilized mannitol containing formulations.

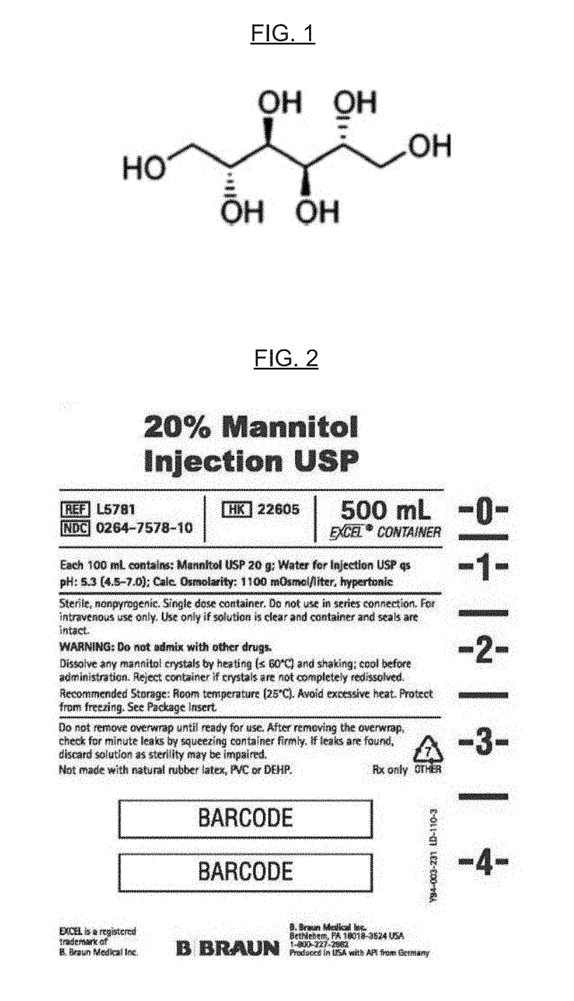

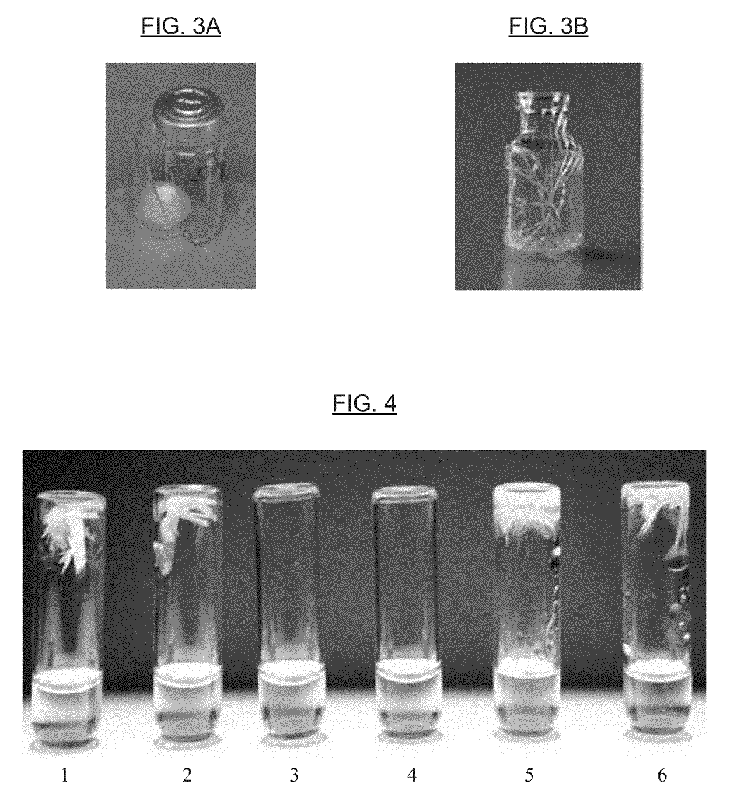

### DETAILED DESCRIPTION

Reference will now be made in detail to various embodiments of the invention, examples of which are illustrated in the accompanying drawings. While the invention will be described in conjunction with the following embodiments, it will be understood that the descriptions are not intended to limit the invention to these embodiments. On the contrary, the invention is intended to cover alternatives, modifications and equivalents that may be included within the spirit and scope of the invention as defined by the appended claims. Furthermore, in the following detailed description, numerous specific details are set forth in order to provide a thorough understanding of the present invention. However, it will be readily apparent to one skilled in the art that the present invention may be practiced without these specific details. In other instances, well-known methods, procedures, components, and circuits have not been described in detail so as not to unnecessarily obscure aspects of the present invention.

The technical proposal(s) of embodiments of the present invention will be fully and clearly described in conjunction with the drawings in the following embodiments. It will be understood that the descriptions are not intended to limit the invention to these embodiments. Based on the described embodiments of the present invention, other embodiments can be obtained by one skilled in the art without creative contribution and are in the scope of legal protection given to the present invention.

Furthermore, all characteristics, measures or processes disclosed in this document, except characteristics and/or processes that are mutually exclusive, can be combined in any manner and in any combination possible. Any characteristic disclosed in the present specification, claims, Abstract and Figures can be replaced by other equivalent characteristics or characteristics with similar objectives, purposes and/or functions, unless specified otherwise.

The present invention relates in part to an effective method of inhibiting crystallization of mannitol (including mannitol analogs and derivatives) using antifreeze proteins (including natural or engineered antifreeze proteins, active fragments of antifreeze proteins, mimetics of antifreeze proteins, their active mimetic fragments, and combinations thereof). The application of this technology also stabilizes a freeze-dried formulation, and in particular, a freeze-dried pharmaceutical formulation including mannitol (and/or mannitol analogs and derivatives). In the present disclosure, a novel and effective method of using an antifreeze protein (AFP) additive to inhibit mannitol crystallization is described. Mannitol crystallization can be suppressed for more than 6 months or for more than a year (e.g., at a temperature of 0-10° C.) by adding an AFP at a concentration as low as 0.0001% w/v.

The present invention has great economic potential. Among other things, it advances methods of stabilizing freeze-dried pharmaceutical formulations. The invention, in its various aspects, will be explained in greater detail below with regard to exemplary embodiments.

Exemplary Methods of Inhibiting or Preventing Crystallization of Sugar Alcohols and/or Sugars

The present method of inhibiting crystallization of mannitol (a sugar alcohol, which can be categorized as a carbohydrate) using an antifreeze protein is similar to, but different from, the method disclosed in U.S. Pat. Appl. Publ. No. 2016/0265073 (the relevant portions of which are incorporated herein by reference). The present method has an effective ratio of the mass of AFP to the volume of mannitol solution of about 0.0001% (w/v) or more (or, alternatively, from about 0.0001% w/v to about 0.001% w/v). In other words, the mass ratio of the antifreeze protein to the carbohydrate can be from about 1:500,000 to about 1:5000 in the present invention, although it can also be different (e.g., from 1:500,000 to 1:10,000, 1:50,000 to 1:1000, or any value or range of values therein). In one example, the crystallization of mannitol was inhibited for more than 12 months.

The term “AFP” may defined herein as the cumulative group consisting of natural or engineered antifreeze proteins, antifreeze polypeptides and antifreeze peptides, active fragments of antifreeze proteins, antifreeze polypeptides and antifreeze peptides, mimetics of antifreeze proteins, antifreeze polypeptides and antifreeze peptides, their active mimetic fragments, and combinations thereof. The foregoing term “antifreeze” may be defined as having or providing one or more thermal hysteresis properties or characteristics. The term “ice-like crystalline structures” may be defined as ice, gas hydrates and Clathrate hydrates that are in the solid phase. The term “critical ratio” may be the molar ratio of additive to AF(G)P compound that completely inhibits the growth of crystals.

In one aspect, the present method utilizes an AFP having a sequence (e.g., AFGP1, AFGP2, AFGP3, AFGP4, AFGP5, AFGP8) of an antifreeze glycoprotein (AFGP) found in the blood of the Antarctic fish Pagothenia borchgrevinki, and variations thereof. The AFGPs from Pagothenia borchgrevinki consist of a repeated sequence of Alanine-Alanine-Threonine and a disaccharide (e.g., β-d-galactosyl(1→3)-α-N-acetylgalactosamine) joined to each threonine through a glycosidic linkage. The different Pagothenia borchgrevinki AGFPs (e.g., AFGP1, AFGP2, AFGP3, AFGP4, AFGP5 and AFGP8) contain different numbers of repeated Ala-Ala-Thr units. For example, AFGP8 includes fewer Ala-Ala-Thr units than the other AFGPs. For convenience, AFGP1, AFGP2, AFGP3, AFGP4 and AFGP5 are often isolated and used as a mixture, which is designated “AFGP1-5.” The Pagothenia borchgrevinki AFGPs are different from the AFP sequences disclosed in U.S. Pat. Appl. Publ. No. 2016/0265073.

The disaccharide has no effect on mannitol crystallization inhibition, as tested in this work. Accordingly, AFPs comprising or consisting of repeated Ala-Ala-Thr units (e.g., having the formula R¹-[Ala-Ala-Thr]ₓ-R², where R¹ is H, C₁₋₆ alkyl, R³—C(═O)— or R³—OC(═O)—, x is an integer of at least 3 [e.g., 3-100, 10-50, or any value or range of values therein], R² is OH, C₁₋₆ alkoxy, R⁴—NH— or R⁴₂N—, R³ is C₁₋₆ alkyl, C₆₋₁₀ aryl, or C₁₋₆ alkyl or C₆₋₁₀ aryl substituted with one or more halogen atoms (e.g., F or Cl) and/or C₁₋₄ alkyl groups, and R⁴ is C₁₋₄ alkyl) are effective in the present invention. Furthermore, other amino acids with similar physical and/or chemical properties may be substituted for one or more of the alanines or threonines in one or more of the units. Accordingly, the AFPs suitable for use in the present method and composition include those having the formula R¹-[AA1-AA1-AA2]ₓ-R², where each AA1 is independently Ala, Asn, Gly, Val, Leu, Pro, Phe, or Ile, (e.g., Ala or Gly), each AA2 is independently Thr or Ser or Tyr, and R¹ and R² are as described above. In some examples, no more than 10% of the alanines or threonines in the formula R¹-[Ala-Ala-Thr]ₓ-R² are replaced with a different amino acid (e.g., Ala replaced with Gly and/or Thr replaced with Ser). Of course, any of the present AFPs may be glycosylated (e.g., with one or more β-d-galactosyl(1→3)-α-N-acetylgalactosamine or other mono- or disaccharide [any of which may include an acetylamido or other C₁₋₄ alkanoylamido groups in place of an OH group] linked to the AA2 hydroxyl group through a glycosidic linkage.

In a further embodiment, the present method and composition can utilize other AFPs, such as DAFPs (including DAFP-1, DAFP-2, DAFP-4 and DAFP-6, disclosed in U.S. Pat. Appl. Publ. No. 2016/0265073 as SEQ ID NOS 1-4), as well as other insect antifreeze proteins. DAFPs have a β-helical or β-solenoid structure. Tenebrio and Dendroides AFPs are hyperactive AFPs with many known isoforms, and their isoforms have high sequence and structural similarity. They are similar to one another, both being hyperactive (i.e., having a relatively high thermal hysteresis value). AFPs are found in different insect families (e.g., beetles, fleas, and moths). Some of these structures share some similarities to Tenebrio and Dendroides AFPs, while others are very different. Insect AFPs have relatively high antifreeze activity.

The AFP may also be selected from other fish AFPs and AFGPs (e.g., type I-IV AFPs and AFGPs), plant AFPs, bacteria AFPs, and fungus AFPs. Type I AFPs include an alanine-rich α-helix. Type II AFPs and type III AFPs are unrelated globular proteins having no repetitive characters (e.g., amino acid sequences). Type II AFPs have a lectin-like fold with mixed α, β, and loop structures, while type III AFPs shows a compact fold with short and/or irregular β-strands. AFGPs are glycoproteins, and may adopt a polyproline II structure. Both O-linked and C-linked analogs of antifreeze glycoprotein have been prepared.

Despite their different structures, the AFPs and AFGPs are thought to have a relatively flat region or surface in their structures (see, e.g., FIG. 6) to recognize ice surfaces. These putative relatively flat surfaces of AFPs may help the AFPs to recognize specific surfaces of the sugar and sugar alcohol crystals.

Type I AFPs can be found in fish such as winter flounder, longhorn sculpin and shorthorn sculpin. Its three-dimensional structure determined. Type I AFPs generally consist of a single, long, amphipathic alpha helix, about 3.3-4.5 kDa in size. There are three faces to the 3D structure: the hydrophobic, hydrophilic, and Thr-Asx faces (see, e.g., FIG. 6). Type III AFPs exhibit similar overall hydrophobicity at ice binding surfaces to type I AFPs. They are approximately 6 kD in size. One type III AFP is fish AFP III, a prototypical globular AFP having size (or molecular mass) of 7 kDa that is present in members of the fish subclass Zoarcoidei. Although the use of DAFP-1 to inhibit crystallization of methyl α-D-mannopyranoside is known, this work extends the crystallization inhibition ability of AFPs to mannitol, mannitol analogs, mannitol derivatives, and other saccharides.

For example, the present AFPs and AFGPs may be useful for inhibiting crystallization of sugars and sugar alcohols of the formula CₘHₙOₚ, where m is an integer of 5 or 6, n is an integer of 2m or 2m+2, and p is equal to m. AFGPs having repeating units of the formula R¹-[AA1-AA1-AA2]ₓ-R², may also be useful for inhibiting crystallization of di- and trisaccharides of the formula CₐH_{b}O_{c}, where a is an integer of 10-12 or 15-18, and b is 2a−2 when a is an integer of 10-12 or 2a−4 when a is an integer of 15-18, and c is a−1 when a is an integer of 10-12 or a−2 when a is an integer of 15-18.

DAFPs, which have more than 13 isoforms, are effective nucleation inhibitors for D-mannitol. FIG. 4 shows six (6) vials 1-6. Vial 1 shows crystals formed from a solution of mannitol alone is distilled deionized water, kept at 4° C. for >12 months. Vial 2 shows crystals formed from a solution of mannitol+denatured DAFP-1 (1% w/v) under identical conditions. Vial 3 shows crystals formed from a solution of mannitol+DAFP-1 (0.001% w/v) under identical conditions. Vial 4 shows crystals formed from a solution of mannitol+DAFP (1% w/v) under identical conditions. Vial 5 shows crystals formed from a solution of mannitol+AFGP1-5 (0.001% w/v) under identical conditions. Vial 6 shows crystals formed from a solution of mannitol+AFGP1-5 (1% w/v) under identical conditions.

Vials 3 and 4 show complete inhibition of mannitol crystallization under conditions representative of those that generally promote mannitol crystallization. Vials 5 and 6 show some inhibition of mannitol crystallization under conditions representative of those that generally promote mannitol crystallization. For example, the sizes of the mannitol crystals in vials 5 and 6 are smaller than those in vials 1 and 2, suggesting that they may be re-dissolved more easily and more quickly than the crystals in vials 1 and 2. In addition, there is a noticeably smaller mass and/or volume of mannitol crystals in vial 6 than in vials 1 and 2.

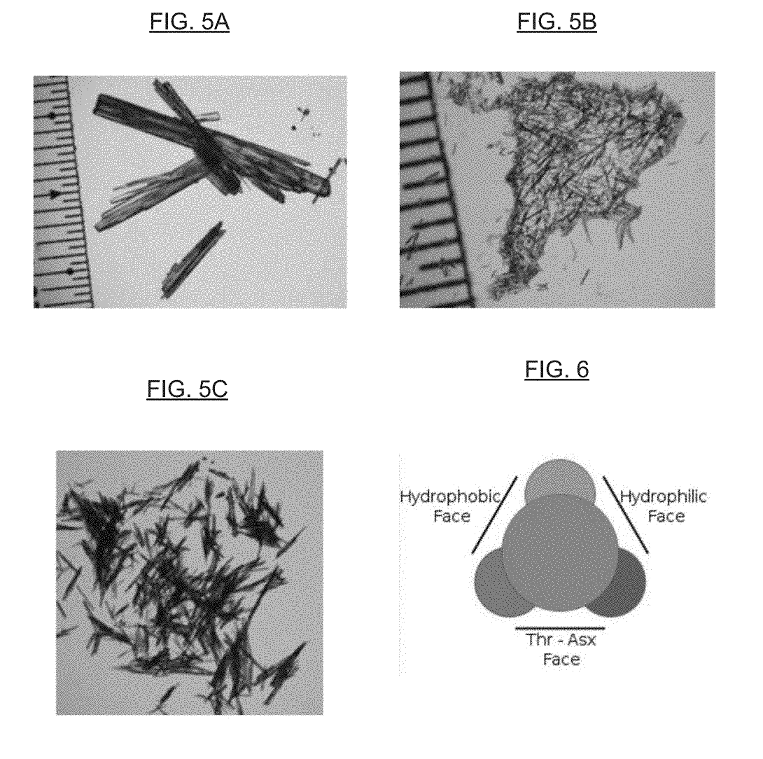

Furthermore, in the presence of mannitol seed crystals, AFPs significant delay the appearance of mannitol crystals and reduce the size of the seed crystals and any subsequently formed crystals. FIG. 5A shows a solution of D-mannitol with seed D-mannitol crystals added thereto (i.e., with no AFP additives added). FIG. 5B shows a solution of D-mannitol with seed D-mannitol crystals added thereto in the presence of DAFP-1 (at a mass ratio of DAFP-1 to mannitol of 5×10⁻⁷:1). FIG. 5C shows a solution of D-mannitol with seed D-mannitol crystals added thereto in the presence of AFPG8 (at a mass ratio of AFPG8 to mannitol of 9×10⁻⁵:1). As can be seen by comparing the sizes of the crystals and the scales in FIGS. 5A-B (in which the scale is identical), the sizes of the mannitol crystals in the presence of DAFP-1 (FIG. 5B) or AFPG8 (FIG. 5C) were much smaller than in the absence of DAFP-1 or AFPG8 (FIG. 5A). The sizes of the mannitol crystals in FIG. 5C are more similar to those in FIG. 5B than those in FIG. 5A.

Materials

All chemicals were purchased from Sigma-Aldrich (St. Louis, Mo.) at GC grade or better except β-galactosyl(1→3)α-N-acetylgalactosamine, which was ordered from Santa Cruz Biotechnology, Inc. (Santa Cruz, Calif.) at analytical grade, and were used without additional purification. Solvents and chemicals for the HPLC experiments were purchased at HPLC grade from Sigma-Aldrich.

All of the aqueous solutions were prepared using Milli-Q water produced from a Synergy water system (Millipore) with a minimum resistivity of 18 MΩ·cm. All of the samples including the proteins and peptide samples were filtered through 0.1 m filters before use, unless otherwise indicated. Sample vials (8 mL, obtained from National Scientific) were used for crystallization experiments. All glassware and stir bars were cleaned as described in Wang et al., “Expanding the molecular recognition repertoire of antifreeze polypeptides: effects on nucleoside crystal growth,” Chem. Commun. 48:11555-11557 (2012).

AF(G)P and Protein Control Preparation

DAFP-1 was prepared as described in Amornwittawat et al., “Effects of polyhydroxy compounds on beetle antifreeze protein activity,” Biochimica et Biophysica Acta (BBA)—Proteins & Proteomics, 1794(2), 341-346 (2009). In general, AFPs can be prepared as described in Wang et al., “Molecular Recognition of Methyl α-D-Mannopyranoside by Antifreeze (Glyco)Proteins,” Journal of the American Chemical Society., 136:8973-8981 (2014). Type I and type III AFPs were purchased from A/F Protein (Waltham, Mass.), which were used as received and/or according to Wang et al., Chem. Commun. 48:11555-11557 (2012). AFGPs were a gift. The amounts of AFPs and denatured DAFP-1 in the stock denatured DAFP-1 were determined by UV-Vis spectroscopy at 280 nm and/or by weight using an analytical balance.

The denatured DAFP-1 was used as a control according to previously disclosed methods (Wang et al., Chem. Commun. 48:11555-11557 [2012]). 1 mM purified DAFP-1 was incubated in 0.10 M sodium citrate and 15.0 mM tris(2-carboxyethyl)phosphine hydrochloride (TCEP) at pH 3.00 for half an hour at 60° C. The final denatured DAFP-1 was further purified using an AKTA Purifier 10 (GE Healthcare) with a Sephacryl S-100 gel filtration column (GE Healthcare). BSA was purchased from Sigma-Aldrich (Item number A7030) and was used as a control. Both BSA and at least one denatured AFP (e.g., denatured DAFP-1) were used as negative controls. The presence of BSA and/or the denatured AFP have no effects on mannitol crystallization.

Crystal Growth Procedure

In the absence of seed crystals, the additive (e.g., the AFP[s], AFGP[s], β-Gal-α-GalNAc, BSA, or denatured DAFP-1) was added to respective sample vials containing 2 mL of supersaturated aqueous D-mannitol solution. The final D-mannitol concentration was about 20% (w/v), and the resulting additive/D-mannitol ratios were 1% (w/v) for β-Gal-α-GalNAc, BSA, and the denatured DAFP-1, and from 0.001% (w/v) to 1% (w/v) for the AFPs/AFGPs, respectively. The vials were gently swirled after the addition, and then closed tightly and kept at 4° C. At least three observations were recorded every day (i.e., at least every 8 hours). The appearance of crystals or crystal growth was observed by unaided eyes and were recorded every 8 hours until no further growth was seen. The experiments were repeated seven times. The crystallization of D-mannitol in the absence of additives (i.e., with no additives) was reproducible with respect to the sizes, shapes and final weights of the crystals.

The crystallization experiments without seed crystals in the vials were stopped after 6 months at 4° C. However, without stopping the experiments, the crystallization in the crystallization experiments without seed mannitol crystals may not be observed for years even at much lower AFP concentrations (e.g., at a mass ratio of the AFP to the carbohydrate is from about 1:15,000).

Vials were turned upside down and then warmed up back to room temperature so that the crystals would not be re-dissolved during the warm-up. Photos of the vials were taken after the warm-up was complete. The solution was then removed from the vials, and crystals (if any) were left at room temperature to dry. Solid-state NMR and FT-IR data for the crystals were taken/recorded before and after the crystals were dried to ensure that there was no change in the crystal form resulting from water evaporation. The weights of the dried crystals were measured and the average values were recorded. The weight of the crystals from the vial containing mannitol alone was the same as the mass of D-mannitol added to the vial.

In the presence of β-form D-mannitol seed crystals, the procedures were the same as the crystallization experiments without the seed crystals above, except that about 1×10⁻⁴% (0.0001%) w/v seed crystals of D-mannitol were added into each vial, and the amount of the additive was from 0.0003% (w/v) to 1% (w/v) on day 1. The vials were gently swirled after the additions and then closed tightly and kept at 4° C. The crystal growth/changes were observed beginning on day 2 for the control and AFGP8 at 0.0003% (w/v). Little or no crystal growth was seen in the vial(s) with DAFP as an additive until day 4, while little growth was seen for the vial(s) with AFGP1-5 as an additive until day 3. After 2 months and one week, the remaining solutions in the vials were quickly removed. The resulting crystals were studied by FT IR and solid-state NMR once the solutions were removed. The optical micrographs of the crystals were taken under a Nikon SMZ-1000 microscope with a DS-Fi2 color camera. The experiments were repeated seven times. The weights of the dried crystals were measured and the average values were recorded.

Solid-State NMR Spectroscopy

Approximately 120 mg of solids were gently ground using a mortar and pestle and packed in a 4 mm wide ZrO₂ rotor with a Kel-F cap. ¹³C-cross polarized magic angle spinning (¹³C CP MAS) solid-state NMR spectra were recorded at 298 K at 75.47 MHz (¹³C) on a Bruker spectrometer using a 4 mm broadband MAS probe with proton broadband decoupler. A spinning frequency of 10 kHz, a CP contact time of 1.5 ms, and a 60 s delay were utilized.

Fourier Transform Infrared (FT-IR) Spectroscopy

FT-IR attenuated total reflectance (ATR) spectra were collected on a Nicolet iS5 FT-IR spectrometer (Thermo Fischer Scientific Inc., Waltham, Mass.) equipped with an iD5 ATR accessory. The IR frequencies were recorded in cm⁻¹, and the spectra were measured in a spectral range from 4,000 to 200 cm⁻¹.

Despite differences in the crystal structures of ice and nucleosides, the molecular recognition repertoire of antifreeze polypeptides (AFPs), which have been demonstrated to efficiently inhibit nucleation of 5-methyluridine, cytidine, and inosine and modify the crystal growth of these nucleosides, has been expanded to inhibit or prevent crystal growth of non-ice-like crystalline solids.

Results and Discussion

The advantageous aspects of the present invention relative to U.S. Pat. Appl. Publ. No. 2016/0265073 include the following. The mass ratio of AFP to mannitol or other carbohydrate is lower (and preferably much lower) than that disclosed in U.S. Pat. Appl. Publ. No. 2016/0265073 to prevent trehalose (or other carbohydrate) crystallization. Also, the type of AFP in the present composition and method is different from the specific AFP sequences disclosed in U.S. Pat. Appl. Publ. No. 2016/0265073.

Effects of Antifreeze Protein on Mannitol Crystallization During Freeze-Drying

Mannitol is a common excipient in lyophilization. However, the crystallization of mannitol may change the distribution of water in the matrix of the freeze-dried product and/or may cause vial breakage, resulting in destabilization of the freeze-dried products. Destabilization is a common industrial issue and has yet to be eliminated. Antifreeze proteins (AFPs) are found in many organisms including bacteria, fish, plants, and insects, and are well-known for their ability to bind to ice crystals and inhibit ice growth. New roles for AFPs in controlling carbohydrate crystallization have been recently reported, where AFPs have been demonstrated to inhibit mannitol crystallization highly effectively.

The effects of AFPs on the crystallization of mannitol during freeze-drying have been investigated, and the resulting freeze-dried solids have been analyzed using polarized microscopy and powder X-ray diffractometry. The results suggest that the presence of AFPs at micro-molar concentrations alters the temperature of ice nucleation and inhibits the crystallization of mannitol during freeze-drying. The powder X-ray diffraction (PXRD) data suggests that AFPs inhibit the mannitol crystals (or the formation thereof) and alters the forms of mannitol crystals (e.g., that do form). The effects of AFPs on mannitol crystallization during freeze-drying is concentration dependent, and are more significant when there is an additional component in the system, such as lactate dehydrogenase. These results suggest applications for AFPs in freeze-drying processes to eliminate deterioration of industrial samples due to crystallization of the excipient(s).

Exemplary Use of AFP in Preventing D-Mannitol Crystallization in Solution

For example, solutions of mannitol may crystallize when exposed to low temperatures (e.g., 4° C.) during storage. Concentrations greater than 15% (e.g., 20% mannitol USP) have a greater tendency to crystallize. The following case shows that by adding tiny amounts of AFPs, the storage of concentrated mannitol solutions can be extended to years at 4° C. (see, e.g., FIG. 4 and Table 1). The presence of tiny amounts of AFPs inhibit the nucleation of mannitol in the solutions in the absence of seed crystals.

TABLE 1
Sample results for D-mannitol crystal growth in the absence of
seed mannitol crystals.

| | Additive:D- | Induction Time^{a} | % Crystal |
|---|---|---|---|
| Additive | Mannitol Ratio | (days) | weights^{b} |
| Pure D-mannitol | n/a | 11 | 100% |
| Gal-N-GalNAc | 1% | 11 | 100% |
| BSA | 1% | 11 | 100% |
| Denatured AFP | 1% | 11 | 100% |
| DAFP | 1 × 10⁻⁶ | Not observed | 0 |
| AFGP1-5 | 2 × 10⁻⁶ | 27 | 33% |
| AFGP8 | 2 × 10⁻⁶ | 24 | 46% |
| Type I AFP | 2 × 10⁻⁶ | 22 | 54% |
| Type III AFP | 2 × 10⁻⁶ | 26 | 39% |

^{a}The day that the first appearance of solid was observed.
^{b}% Crystal weights: mannitol crystals in the D-mannitol samples were grown in the absence of seed mannitol crystals for 6 months at 4° C., then were dried. The weights of these crystals were measured and compared to the amounts of D-mannitol in the solutions.

In the presence of seed mannitol crystals, AFPs highly effectively inhibit the crystal growth of mannitol (see, e.g., FIGS. 5B and 5C). Our results suggest interactions between an AFP and mannitol.

FIG. 7A shows the CP MAS ¹³C solid-state NMR spectrum of D-mannitol in the presence of seed β form D-mannitol crystals. FIG. 7B shows the CP MAS ¹³C solid-state NMR spectrum of D-mannitol in the presence of seed β form D-mannitol crystals and an AFP. The weight of the seed β form D-mannitol crystals added to the sample is 2 Gig, and the weight ratio of the AFP to the total D-mannitol in the sample is about 1×10⁻⁶:1.

The broadening peaks of D-mannitol in the presence of an AFP are observed in FIG. 7B, suggesting that the presence of the AFP inhibits the crystallization of D-mannitol and results in the formation of amorphous mannitol. The results also suggest interactions between D-mannitol and AFP (see, e.g., Morin et al., Variable-Temperature Solid-state 13C NMR Studies of Nascent and Melt-Crystallized Polyethylene, Macromolecules 1995, 28, 3248-3252). Such peak broadening effects were not observed in the negative controls used in this study (i.e., Gal-N-GalNAc, BSA and the denatured AFP). There is no difference in the CP MAS ¹³C solid-state NMR spectra between D-mannitol and D-mannitol in the presence of the negative controls.

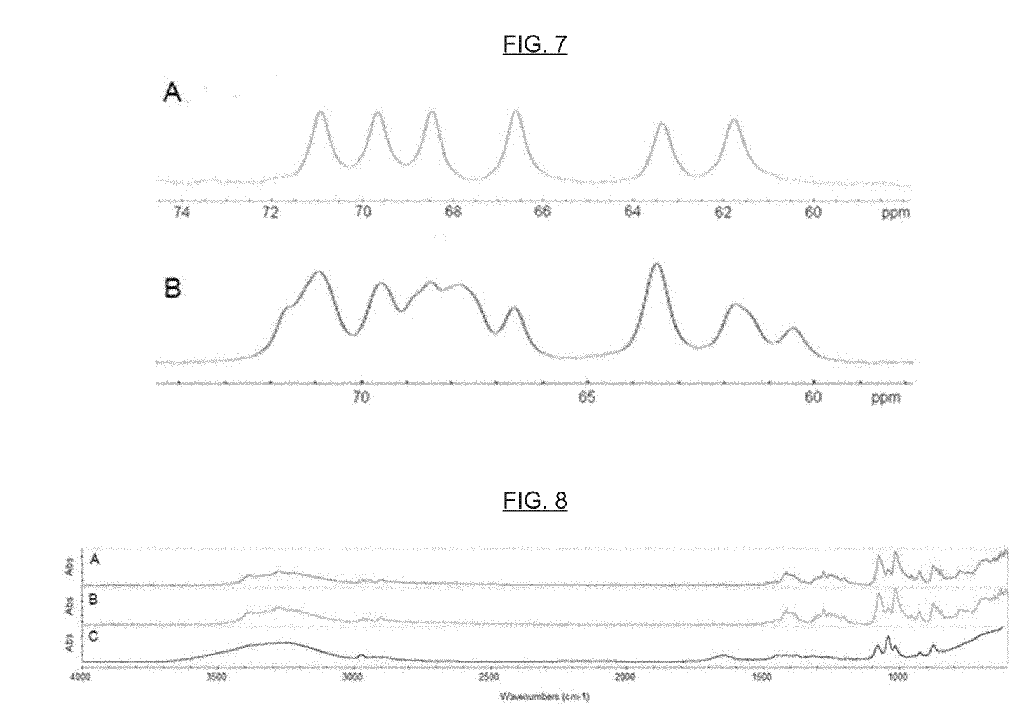

FIGS. 8A-C show the FT-IR spectrum of D-mannitol in the presence of seed β form D-mannitol crystals (FIG. 8A), D-mannitol in the presence of seed β form D-mannitol crystals and a negative control (FIG. 8B), and D-mannitol in the presence of seed β form D-mannitol crystals and an AFP (FIG. 8C). The weight of the seed of β form D-mannitol crystals added to the sample is 2 μg, and the ratio by weight of the AFP to the total D-mannitol in the sample is about 1×10⁻⁶:1.

The loss of FT-IR band intensity between 1200-1500 cm⁻¹ was observed in the D-mannitol samples in the presence of seed β form D-mannitol crystals and an AFP, indicating the presence of amorphous mannitol in the samples.

Exemplary Use of AFP in Preventing Crystallization in Freeze-Dried Formulations Using Mannitol During Freeze-Drying and in Subsequent Storage

The loss of activity of freeze-dried bioactive reagents (e.g., probiotics, microbes, proteins, RNA, DNA, cells, tissues, and biologics) is directly related to the degree of crystallinity of the cryoprotective molecules in the formulations. Therefore, the crystallization of the excipients should be avoided in the formulation of bioactive reagents (see, e.g., U.S. Pat. No. 6,284,277 B1, entitled “Stable Freeze-Dried Pharmaceutical Formulation,” the relevant portions of which are incorporated herein by reference, and the references cited in this patent, including U.S. Pat. Nos. 4,537,883, 5,558,880 and 5,885,486, the relevant portions of which are incorporated herein by reference, European Pat. Publ. Nos. 394 045 and 682 944, the relevant portions of which are incorporated herein by reference, Great Britain Pat. Publ. No. 2 021 581, the relevant portions of which are incorporated herein by reference, PCT Pat. Publ. No. WO 1993/23017, the relevant portions of which are incorporated herein by reference, and Izutsu K. L., Yoshioka S., Terao T., Decreased protein-stabilizing effects of cryoprotectants due to crystallization, Pharm. Research., 1993, vol. 10, No. 8, 1232-1237, and Hermansky M., Pesak M., Lyophilization of drugs, VI: Amorphous and Crystalline Forms, Cesk. Farm., 1993, 42(2), 95-98, the relevant portions of which are incorporated herein by reference).

The effects of AFPs on mannitol crystallization during freeze-drying as well as on the protection of a model protein, lactate dehydrogenase (LDH), have been investigated. LDH is involved in the reversible transformation of pyruvate (the end product of glycolysis) to lactate. It is known that crystalline excipients destabilize LDH in formulations (see, e.g., Izutsu et al., Increased Stabilizing Effects of Amphiphilic Excipients on Freeze-Drying of Lactate Dehydrogenase (LDH) by Dispersion into Sugar Matrices [1995], 12:6, 838-843).

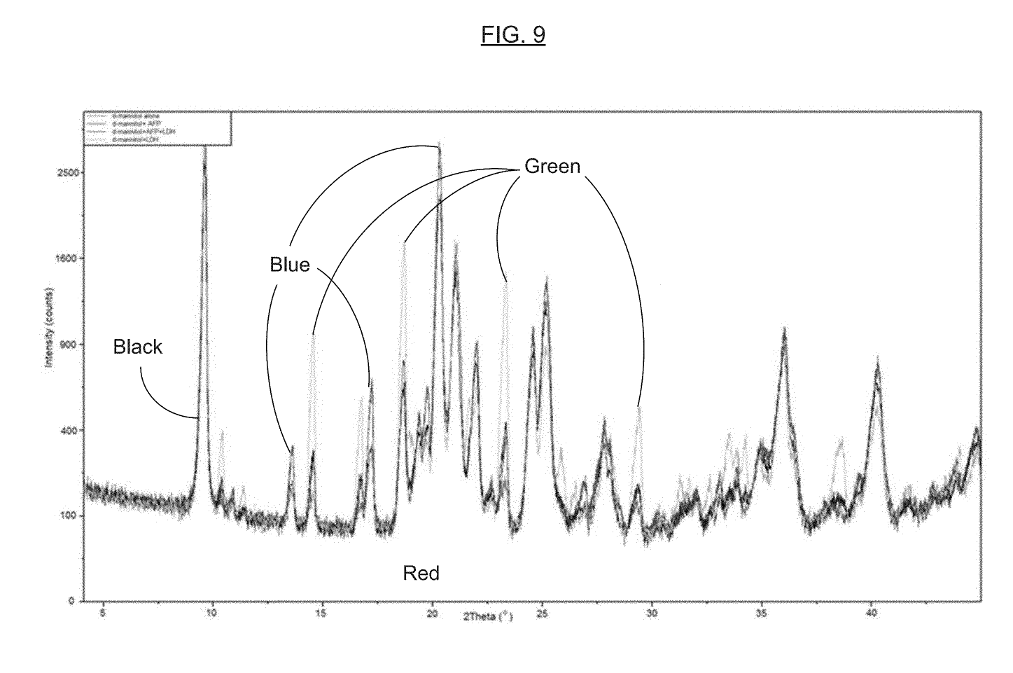

The freeze-drying experiments were performed with fast freezing (1° C./min) to −45° C., then holding the temperature at −23° C. for an hour (i.e., annealing), then maintaining the sample on a shelf at 35° C. for 16 h. Some of the freeze-dried samples were analyzed using powder X-ray diffraction (pXRD). The pXRD data were collected at room temperature on an Empyrean powder X-ray diffractometer using Cu Kα radiation. The remaining freeze-dried samples were stored at room temperature for 3 months. Then, the activity of LDH in the absence and presence of the AFP was measured according to a previously published protocol (see, e.g., Bergmeyer, H. U., and Bernt, E., Methods of Enzymatic Analysis, Volume II; 2nd ed.; Bergmeyer, H. U., ed.; Academic Press, New York, N.Y. [1974], 574-579), with inconsequential modifications. Activity of LDH was detected in the sample of LDH with mannitol and AFP, while little activity was found for the LDH in the sample with mannitol alone. The presence of LDH seems to induce a degree of crystallinity of mannitol in the freeze-dried powders (see, e.g., the light green line in FIG. 9), while the presence of an AFP in the sample of mannitol and LDH greatly reduces the degree of crystallinity of mannitol (see, e.g., the black line in FIG. 9). The reduction of crystalline mannitol in the freeze-dried samples with the AFP prevents the denaturation of the bioactive substance in the formulations and stabilize the bioactive substance during storage. The suppression of mannitol crystallization in the formulations during freeze-drying in the presence of an AFP is more significant at a ratio of mannitol to AFP of about 2,000:1 as detected using pXRD.

The red line in FIG. 9 is an X-ray powder diffraction (pXRD) pattern of a lyophilized formulation of D-mannitol, the blue line in FIG. 9 is a lyophilized formulation of D-mannitol and DAFP-1, the black line in FIG. 9 is a lyophilized formulation of D-mannitol, LDH, and the AFP, and the light green line in FIG. 9 is a lyophilized formulation of D-mannitol with LDH. The weight ratio between mannitol and the AFP (about 20,000:1) is the same in the relevant samples. The percentage of mannitol (7%) is the same in all samples, and the amount of LDH (25 μg/mL) is the same in the relevant samples.

### CONCLUSION/SUMMARY

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

1. A composition, comprising:
an antifreeze protein (AFP), an analog thereof, an active fragment, a mimetic thereof, an antifreeze peptoid, or an antifreeze polymer; and
a carbohydrate selected from the group consisting of sugars and sugar alcohols of the formula CₘHₙOₚ, where m is an integer of 5 or 6, n is an integer of 2m or 2m+2, and p is equal to m, and di- and trisaccharides of the formula CₐH_{b}O_{c}, where a is an integer of 10-12 or 15-18, and b is 2a−2 when a is an integer of 10-12 or 2a−4 when a is an integer of 15-18, and c is a−1 when a is an integer of 10-12 or a−2 when a is an integer of 15-18;
wherein the AFP and the carbohydrate are present in a mass ratio of from about 1:500,000 to about 1:100.

2. The composition of claim 1, wherein the AFP is selected from the group consisting of natural and engineered AFPs, antifreeze polypeptides and antifreeze peptides, active fragments of antifreeze proteins, antifreeze polypeptides and antifreeze peptides, mimetics of antifreeze proteins, antifreeze polypeptides and antifreeze peptides, active fragments of antifreeze protein, polypeptide and peptide mimetics, and combinations thereof.

3. The composition of claim 1, wherein the AFP has an amino acid sequence of the formula R¹-(AA1-AA1-AA2)ₓ-R², where each AA1 is independently Ala, Asn, Gly, Val, Leu, Pro, Phe, or Ile, each AA2 is independently Thr or Ser or Tyr, R¹ is H, C₁₋₆ alkyl, R³—C(═O)— or R³—OC(═O)—, x is an integer of at least 3, R² is OH, C₁₋₆ alkoxy, R⁴—NH— or R⁴₂N—, R³ is C₁₋₆ alkyl, C₆₋₁₀ aryl, or C₁₋₆ alkyl or C₆₋₁₀ aryl substituted with one or more halogen atoms and/or C₁₋₄ alkyl groups, and R⁴ is C₁₋₄ alkyl.

4. The composition of claim 3, wherein the amino acid sequence has the formula R¹-[Ala-Ala-Thr]ₓ-R², where 0-10% of the alanines in the formula R¹-[Ala-Ala-Thr]ₓ-R² are replaced with glycine or leucine and 0-10% of the threonines in the formula R¹-[Ala-Ala-Thr]ₓ-R² are replaced with serine or tyrosine.

5. The composition of claim 1, wherein the carbohydrate is the sugar or sugar alcohol of the formula CₘHₙOₚ or the analog or derivative of the sugar or sugar alcohol.

6. A formulation comprising the composition of claim 1 in an acceptable excipient.

7. The formulation of claim 6, further comprising a pharmacologically active or biologically active substance or compound.

8. A composition, comprising:
an antifreeze protein (AFP), an analog thereof, or an active fragment or mimetic thereof; and
a sugar or sugar alcohol of the formula CₘHₙOₚ or an analog or a derivative thereof, where m is an integer of 5 or 6, n is an integer of 2m or 2m+2, and p is equal to m;
wherein the AFP and the sugar or sugar alcohol are present in a mass ratio of from about 1:500,000 to about 1:1,000.

9. The composition of claim 8, where the insect AFP is has an amino acid sequence of the formula A-(Thr-X-Thr-Y)_{z}-B, where A is a sequence of at least 1 amino acid, X is a sequence of from 1 to 3 amino acids, Y is a sequence of 1-12 amino acids, z is an integer of from 3 to 32, and B is a sequence of 1-5 amino acids.

10. The composition of claim 8, where the insect AFP is DAFP-1 (SEQ ID NO: 2), DAFP-2 (SEQ ID NO: 3), DAFP-4 (SEQ ID NO: 4) or DAFP-6 (SEQ ID NO: 5).

11. A formulation comprising the composition of claim 8 in an acceptable excipient.

12. The formulation of claim 11, further comprising a pharmacologically active or biologically active substance or compound.

13. A composition, comprising:
an antifreeze protein (AFP), an analog thereof, an active fragment, a mimetic thereof, an antifreeze peptoid, or an antifreeze polymer; and
a carbohydrate selected from the group consisting of mannitol, analogs of mannitol, and derivatives of mannitol,
wherein the AFP and the carbohydrate are present in a mass ratio of from about 1:500,000 to about 1:100.

14. A method of inhibiting or preventing crystallization of a carbohydrate, comprising:
combining the composition of claim 13 in a mass ratio of the AFP to the carbohydrate of from about 1:500,000 to about 1:100 to form a mixture; and
storing the mixture at a temperature at which the carbohydrate can crystallize in the absence of the AFP for a minimum length of time.

15. A method of freeze-drying a formulation, comprising:
the method of claim 14;
combining a pharmacologically active or biologically active substance or compound with the mixture and an acceptable excipient such that, after performing the method of claim 14 and combining the pharmacologically active or biologically active substance or compound, a solution is formed; and
freeze-drying the solution to form the formulation.

16. A method of inhibiting or preventing crystallization of a carbohydrate, comprising:
combining the composition of claim 13 in a mass ratio of the AFP to the sugar or sugar alcohol of from about 1:500,000 to about 1:1000 to form a mixture; and
storing the mixture at a temperature at which the sugar or sugar alcohol can crystallize in the absence of the insect AFP for a minimum length of time.

17. A method of freeze-drying a formulation, comprising:
the method of claim 16;
combining a pharmacologically active or biologically active substance or compound with the mixture and an acceptable excipient such that, after performing the method of claim 16 and combining the pharmacologically active or biologically active substance or compound, a solution is formed; and
freeze-drying the solution to form the formulation.

18. The composition of claim 13, wherein the AFP has an amino acid sequence of the formula R¹-(AA1-AA1-AA2)ₓ-R², where each AA1 is independently Ala, Asn, Gly, Val, Leu, Pro, Phe, or Ile, each AA2 is independently Thr or Ser or Tyr, R¹ is H, C₁₋₆ alkyl, R³—C(═O)— or R³—OC(═O)—, x is an integer of at least 3, R² is OH, C₁₋₆ alkoxy, R⁴—NH— or R⁴₂N—, R³ is C₁₋₆ alkyl, C₆₋₁₀ aryl, or C₁₋₆ alkyl or C₆₋₁₀ aryl substituted with one or more halogen atoms and/or C₁₋₄ alkyl groups, and R⁴ is C₁₋₄ alkyl or the formula A-(Thr-X-Thr-Y)_{z}-B, where A is a sequence of at least 1 amino acid, X is a sequence of from 1 to 3 amino acids and which may include Cys, Y is a sequence of 1-12 amino acids, z is an integer of from 3 to 32, and B is a sequence of 1-5 amino acids.


SEQUENCE LISTING (5 sequences)

SEQ ID NO: 1 - protein, 11 aa, Dendroides canadensis
QCTGGSDCSSC

SEQ ID NO: 2 - protein, 83 aa, Dendroides canadensis
NCTGGSDCRSCTVSCTDCQNCPNARTACTRSSNCINALTCTDSYDCHNAETCTRSTNCYKAKTCTGSTNCYEATACTDSTGCP

SEQ ID NO: 3 - protein, 84 aa, Dendroides canadensis
QCTGGSDCRSCTVSCTDCQNCPNARTACTRSSNCNNALTCTDSYDCHNAETCTRSTNCYKAKTCTGSTNCYEATTACTDSTGCP

SEQ ID NO: 4 - protein, 71 aa, Dendroides canadensis
QCTGGSDCQSCTVSCTDCQNCPNARTACTGSSNCINALTCTDSHDCHNAETCTRSTNCYKAKTCTDSTGCP

SEQ ID NO: 5 - protein, 71 aa, Dendroides canadensis
QCTGGSDCSSCTVSCTNCQNCPNARVACTGSTNCINALTCTDSHDCHNAETCTRSTNCYKAKTCTDSTGCP